(19) United States
(12) Patent Application Publication
(10) Pub. No.: US 2019/0021873 A1
(43) Pub. Date: Jan. 24, 2019

(54) CONTINUOUSLY ADJUSTABLE INTERVERTEBRAL IMPLANT

(71) Applicant: LinkSPINE, Inc., CoLumbia, MD (US)

(72) Inventor: Klaus DMUSCHEWSKY, Hamburg (DE)

(73) Assignee: LinkSPINE, Inc., Columbia, MD (US)

(21) Appl. No.: 16/062,836

(22) PCT Filed: Dec. 15, 2015
(86) PCT No.: PCT/EP2015/079909

§ 371 Date: Jun. 15, 2018

(51) Int. Cl. A61F 2/44

(52) U.S. Cl. CPC A61F 2/4425; A61F 2002/30331

(57) ABSTRACT

An intervertebral implant comprising at least two mounting surfaces facing away from each other, for mounting on facing vertebral body surfaces of two adjacent vertebrae, a distance between the two mounting surfaces being modifiable by means of an expansion device arranged between the mounting surfaces and comprising at least one actuating element, the expansion device comprising at least two pivotable lever elements which are connected to each other by means of a rotational shaft in a scissor-type manner, at least one widening component configured to open up the lever elements, and at least one securing element, the securing element configured to fix the minimum widening of the lever elements. The structure of the intervertebral implant provides a high load-bearing capacity and a high level of strength development in addition to a sufficiently large total height adjustment during the expansion thereof. Furthermore, a lordosis angle can be adjusted.

### REFERENCE TO RELATED APPLICATIONS

This application is a national stage application under 35 USC 371 of International Application No. PCT/EP2015/079909, filed Dec. 15, 2015, the entire contents of which are incorporated herein by reference.

### FIELD OF THE INVENTION

The invention relates to an intervertebral implant.

### BACKGROUND OF THE INVENTION

The intervertebral disks of the spinal columns may suffer degeneration as a result of wear or of pathological changes. If conservative treatment by medication and/or physiotherapy is ineffective, surgical treatment is sometimes indicated. In this connection, it is known for a movable or immovable implant to be inserted into the intervertebral space containing the degenerated intervertebral disk. This implant takes over the support function of the degenerated intervertebral disk. An immovable implant is also referred to as a “fusion implant” since it prevents a relative movement of the adjacent vertebrae.

Various surgical techniques are known for implanting a fusion implant. The access to the intervertebral disk can be from the ventral direction, in order thereby to avoid the danger of damaging the spinal cord. However, this necessitates a long access route through the abdominal cavity or thoracic cavity of the patient. Since this can cause complications, an alternative access route has become established, namely from the dorsal direction. These surgical techniques from the immediate dorsal direction, or more from the side, are known as posterior lumbar intervertebral fusion (PLIF) or transforaminal lumbar interbody fusion (TLIF), in which the intervertebral disk is exposed from the posterior or lateral direction, respectively. Because of the small transverse incisions used in minimally invasive surgery, the size of the fusion implants is greatly restricted.

The implantation of intervertebral implants by PLIF takes place in a very sensitive region near the spinal cord, where nerve endings emerge from the spinal canal of the vertebral column. An operation therefore generally poses the risk of damaging these nerve endings. For this reason, openings that are as small as possible are created for implantation by minimally invasive surgery, the consequence of which is that the intervertebral implants have to be configured as small as possible. Moreover, the intervertebral implants, serving to replace the intervertebral disk, are intended either to cover the greatest possible surface area or separate the vertebrae from each other or to establish a fixed angle between the vertebrae and support the vertebrae.

Intervertebral implants therefore have expansion devices. The expansion devices can, for example, increase the distance between the contact faces of the implant on adjacent vertebrae. Alternatively, they can increase the size of the contact faces of an implant on the vertebrae.

It is known to provide locking mechanisms for expansion of the intervertebral implants. Locking mechanisms permit stepped expansion of the intervertebral implant. A continuous adjustment of the intervertebral implant, for flexible and precise height adjustment, is not possible with locking mechanisms.

A continuous adjustment can be carried out by means of worm gears in combination with movement threads or the traction wedge technique. These devices have the disadvantage that they only meet individual requirements of the mechanics of the implants. These requirements include a high load-bearing capacity, a high level of force development during the expansion of the implant, and the possibility of adjusting a lordosis angle. If the lordosis angle can be adjusted, then the adjustment of the overall height of the implant is lacking. Mechanisms with a high load-bearing capacity for supporting the vertebrae have large dimensions, which prevent their use in a PLIF procedure.

### SUMMARY OF THE INVENTION

An object of the invention is therefore to make available a device that avoids the abovementioned disadvantages.

In an intervertebral implant comprising at least two opposite contact faces, directed away from each other, for placement on mutually facing vertebral body surfaces of two adjacent vertebrae, wherein a distance between the two contact faces is modifiable by means of an expansion device which is arranged between the contact faces and has at least one actuating element, provision is made, according to aspects of the invention, that the expansion device has at least two pivotable lever elements which are connected to each other like scissors via a pivot pin, and the expansion device comprises at least one widening component, which is actuated by means of the actuating element and is designed to spread open the lever elements, and at least one securing element, wherein the securing element is designed to fix the minimal spreading-open of the lever elements.

A number of terms will first be explained in more detail:

Spreading open is understood as the pivoting of the lever elements from a closed state, corresponding to that of a closed pair of scissors, to a spread-open state, corresponding to that of an opened pair of scissors.

A minimal spreading-open is understood as a spreading-open of the lever elements that cannot be further reduced. That is to say, the lever elements cannot be transferred from the spread-open state to the closed state any further than the minimal spreading-open.

The invention is based on the concept of bringing about an expansion of the intervertebral implant by means of the scissor mechanism, which utilizes the lifting platform principle. This principle is realized by the lever elements connected to each other like scissors via a pivot pin. The intervertebral implant has a low height in the closed state, such that it can be inserted between the vertebrae through a small opening by means of minimally invasive surgery. In this state, the scissor mechanism is closed. When the intervertebral implant is arranged at its intended position, its expansion can be effected by the spreading-open of the lever elements to the spread-open state.

The spreading-open of the lever elements is effected by actuation of a widening component by the actuating element. When actuated, the widening component applies a torque to the lever elements. On account of the torque, the lever elements are pivoted about the pivot pin and spread open like scissors. The spreading-open of the lever elements has the effect that the distance between the contact faces increases. At the end of the pivoting movement, the securing element fixes the minimal spreading-open of the lever elements. As long as the actuating element is not actuated, the spreading-open of the lever elements can be reduced only as far as the minimal spreading-open set by the securing element.

The widening component is able to exert a considerable torque on the lever elements. Sufficient force can thus be applied to space the two vertebrae apart. Moreover, the contact faces and the lever elements can be loaded by considerable forces. The scissor mechanism of the expansion element can thus withstand the load exerted by the vertebral column.

The invention moreover permits adaptation of a lordosis angle between adjacent vertebrae, by means of adapting the length of the lever arms of the lever element. A lever arm is understood as the section of the lever element that is arranged between the pivot pin and an end portion of the lever element. In the closed state of the implant, lever arms of the two lever elements bearing on each other are of equal length. However, the ratio of the lengths of the lever arms of a single lever element can vary, such that the mutually opposite contact faces of the intervertebral implant are pivoted relative to each other when the lever elements are spread open.

The intervertebral implant according to the invention can be used to adjust the height between adjacent vertebrae.

Alternatively, the intervertebral implant is designed to increase, by expansion, the bearing surface area that it spans between adjacent vertebrae. The respective alternative is defined by the orientation of the implant when inserted into the intervertebral space.

Both alternatives provide fusion of the adjacent vertebrae. These vertebrae can no longer move relative to each other after the implantation and they form one unit during a movement of the vertebral column.

In a first advantageous embodiment, the pivot pin divides the lever elements into two lever arms of equal length. This makes it possible to lift the vertebrae with the vertebral body surfaces oriented parallel to each other.

In a second and alternative advantageous embodiment, the pivot pin divides the lever elements into two lever arms of different length. This embodiment permits a structural adjustment of a lordosis angle between the vertebrae.

Advantageously, the securing element and the actuating element are formed in one piece. The securing element is expediently designed as a screw and the actuating element is designed as the head of the screw. The widening component is designed as at least two wedges connected to the screw as a spindle drive. The screw forms the spindle drive in combination with the threads of the wedges. Spindle drives are self-locking, such that an adjustment of the wedges can be carried out only by actuation of the head of the screw. In this embodiment, the widening components are thus secured automatically. The wedges are arranged on opposite sides of the pivot pin between the lever elements. The wedges each have a wedge tip, the latter pointing in opposite directions. With this arrangement, the widening elements are moved relative to each other along the screw when the screw is rotated via the actuating element. The wedges are pressed with their wedge tips between the lever elements. This results in a spreading-open of the lever elements which are connected like scissors.

Advantageously, at least one contact face is arranged on a bridge element which is arranged on one lever element by means of a fixed bearing, wherein the bridge element is arranged on the other lever element by means of a floating bearing. In contrast to a contact face that can be arranged on the lever elements themselves, the contact face arranged on the bridge element maintains its size during the spreading-open of the lever elements. This permits a uniform distribution of the pressure on the vertebral body surfaces. Moreover, a maximum bearing surface area is made available, since the bridge element spans the free space between the lever elements that arises as a result of the spreading-open of the lever elements.

Advantageously, the floating bearing is designed as a slideway arranged on one lever element. The floating end of the bridge element slides along the slideway during the spreading-open of the lever elements. In this way, the friction between the lever element and the bridge element is reduced, such that the spreading-open movement can take place more easily and damage to the lever element or the bridge element can be avoided.

Advantageously, the bridge elements, in the closed state, lie flush on the intervertebral implant. Moreover, the lever elements and the bridge elements, in the closed state, form at least one closed lateral face of the intervertebral implant. The surface of the intervertebral implant has small openings. The intervertebral implant thus has a streamlined shape which simplifies the insertion of the intervertebral implant into the intervertebral space.

It is moreover expedient that the lateral face of the intervertebral implant, in the spread-open state, has apertures. Filler substances that promote the growth of bone through the intervertebral implant are introduced into these apertures.

It is expedient that the contact faces have a structured surface. By virtue of the structured surfaces, the contact faces have an enhanced hold on the vertebral body surfaces. The implant is thus prevented from slipping off or slipping away.

Moreover, a part of the contact face is expediently arranged at the end regions of the lever elements and, in the closed state, is inclined about a central axis of the intervertebral implant. The angle of the inclination of the part of the contact face is advantageously 5° to 30°, preferably 10° to 20°, more preferably 15°. In this way, a parallel orientation of the contact face with respect to the vertebral body surfaces is obtained in the spread-open state. The vertebral body surface thus bears securely on the contact faces.

Moreover, the intervertebral implant advantageously comprises a strike face, which is designed to take up hammer blows. The implant can thus be inserted into the intervertebral space by means of a hammer.

The intervertebral implant is advantageously made of titanium, a titanium alloy, CoCr or PEEK. These materials avoid complications and rejections by patients.

The invention moreover relates to a set of intervertebral implants, wherein the intervertebral implants differ in terms of the length ratio of the two lever arms of a lever element.

At least one intervertebral implant can be configured as per at least one of the features of the above description.

### BRIEF DESCRIPTION OF THE DRAWINGS

The invention is explained in more detail with reference to the attached drawing, which shows advantageous embodiments. In the drawings:

FIGS. 1a, 1b and 1c each show a schematic view of an intervertebral implant, (la) in the closed state, (1b) in the spread-open state, and (1c) with a lordosis angle adjustment, according to some embodiments;

FIGS. 2a, 2b and 2c each show a schematic view of components of an intervertebral implant, according to some embodiments; and

FIGS. 3a and 3b each show a schematic view of an intervertebral implant inserted in the intervertebral space, (3a) in the closed state, and (3b) in the spread-open state, according to some embodiments.

### DETAILED DESCRIPTION OF THE INVENTION

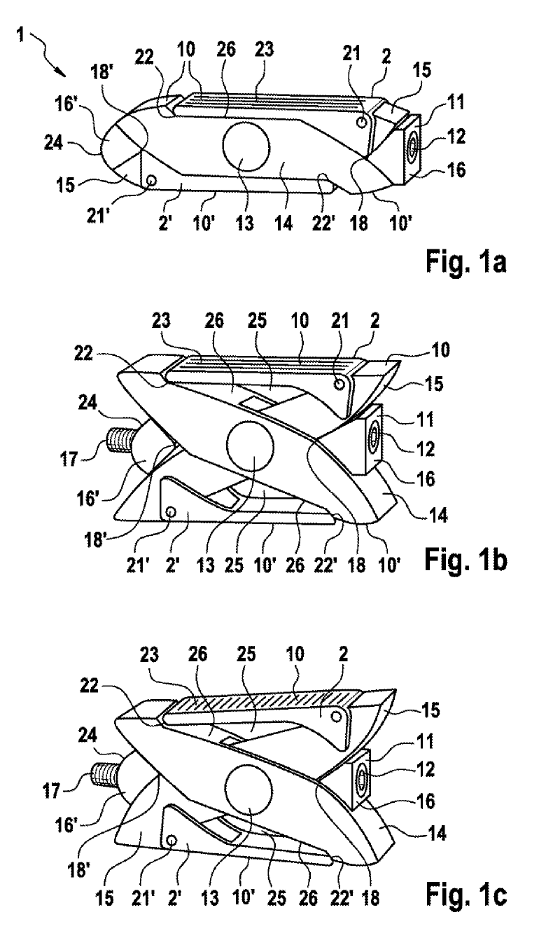

The intervertebral implant, according to one embodiment, is designated in its entirety by reference sign 1. It comprises two lever elements 14, 15 which are connected to each other like scissors by means of a pivot pin 13. The intervertebral implant 1 is shown in the closed state in FIG. 1a. The pivot pin 13 divides both lever elements 14, 15 into halves of equal size. Each of the halves is a lever arm. In the embodiments described here, lever arms of different lever elements 14, 15 are of equal length in the closed state. However, they can also have different lengths.

Between lever arms arranged next to each other in the closed state of the lever arms 14, 15, widening components 16, 16′ are arranged on mutually opposite sides of the intervertebral implant 1. The widening components 16, 16′ can be moved toward each other by means of an actuating element 12. They are moved toward the pivot pin 13 and pressed farther between the lever arms of the lever elements 14, 15. The actuating element 12 is arranged on one of the widening components 16, 16′.

The widening components 16, 16′ are wedge-shaped. The wedge tips 18, 18′ are oriented toward the respective other widening component 16, 16′ and point farther between the lever elements 14, 15. The side walls of the widening components 16, 16′ bearing on the wedge tips 18, 18′ bear on the lever elements 16, 16′. By means of the side walls, the widening components 16, 16′ apply a torque to the lever elements 14, 15 during insertion between the lever elements 14, 15. The torque causes the lever elements 14, 15 to spread open. The spread-open state of the intervertebral implant 1 shown in FIG. 1a is depicted in FIG. 1b.

Contact faces 10, 10′ for placement on vertebral body surfaces 30, 30′ of adjacent vertebrae 3, 3′ are arranged at the end portions of the lever arms 14, 15. In the spread-open state according to FIG. 1b, the contact faces 10, 10′ are spaced farther apart from each other compared to the closed state according to FIG. 1a.

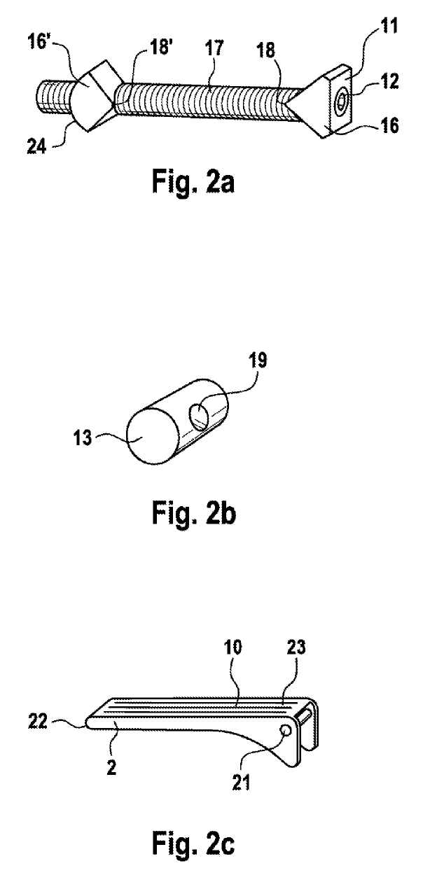

The widening components 16, 16′ have inner threads. As is shown in FIG. 2a, they are connected to each other via a screw, which functions as securing element 17. The inner threads and the screw form a spindle drive. The screw has a head, which functions as actuating element 12. An actuation of the actuating element 12 causes a rotation of the screw, as a result of which the widening components 16, 16′ can be moved toward each other or away from each other. Without rotation of the screw as securing element 17, the distance between the widening components 16, 16′ does not change, on account of the self-locking of the spindle drive, which fixes the position of the widening components 16, 16′. The minimal spreading-open of the lever elements 14, 15 is thus set by the securing element 17. The widening components 16, 16′ remain at their position between the lever elements 14, 15. In combination with the pressure of the vertebrae 3, 3′ on the contact faces 10, 10′, which pressure causes a torque that transfers the intervertebral implant 1 from the spread-open state to the closed state, further pivoting of the lever elements 14, 15 is prevented.

According to FIG. 2b, the pivot pin 13 of the lever elements 14, 15 has a guide bore 19. The securing element 17 is guided through this bore 19. The widening components 16, 16′ are arranged on the securing element 17 on the opposite sides of the pivot pin 13. With the guide bore 19, the widening components 16, 16′, the securing element 17 and the actuating element 12 are guided between the lever elements 14, 15.

FIG. 1c shows an embodiment of an intervertebral implant 1 whose lever elements 14, 15 are divided by the pivot pin 13 into lever arms of different sizes. When the lever elements 14, 15 are spread open, the end portions of the longer lever arms are spaced farther apart from each other than are the end portions of the short lever arms. This results in an angle between the contact faces 10, 10′. With this embodiment of the intervertebral implant 1, a lordosis angle can be adjusted between adjacent vertebrae 3, 3′.

The intervertebral implant 1 moreover has bridge elements 2, 2′. An example of a bridge element 2, 2′ is shown in FIG. 2c. The bridge elements 2, 2′ comprise a part of the contact faces 10, 10′. They are connected rotatably to one of the lever elements 14, 15 via fixed bearings 21, 21′. They are supported on the respective other lever element 14, 15 via a floating bearing 22, 22′. The floating bearing 22, 22′ is formed by a slideway 26, which is arranged on the respective other lever element 14, 15. When the intervertebral implant 1 is spread open, the bridge elements 2, 2′ slide along the respective lever element 14, 15 via the floating bearing 22, 22′ designed as slideway 26. In doing so, the bridge elements 2, 2′ are rotated about the fixed bearings 21, 21′. The bridge elements 2, 2′ thus span the distance between the lever elements 14, 15. Apertures 25 thus arise in the lateral face of the intervertebral implant 1. Substances that promote bone growth can be introduced into these apertures.

The size of the contact faces 10, 10′ arranged on the bridge elements 2, 2′ is not altered by the expansion procedure, in contrast to the contact faces 10, 10′ arranged on the lever elements 2, 2′. A maximum contact face 10, 10′ is thus made available to the vertebral body surfaces 30, 30′ of the vertebrae 3, 3′.

The contact faces 10, 10′ have structured surfaces 23. The contact to the vertebral surface bodies 30, 30′ of the vertebrae 3, 3′ is improved by means of the structures surfaces 23. Moreover, the structured surface 23 avoids the intervertebral implant 1 slipping on the respective bone surface. The structure of a structured surface 23 can comprise transverse grooves, longitudinal grooves or spikes.

Moreover, in the closed state of the intervertebral implant 1, the part of the contact faces 10, 10′ that is arranged at the ends of the lever elements 14, 15 is inclined by 15° with respect to a central axis of the intervertebral implant 1. During the transfer of the intervertebral implant 1 to the spread-open state, the contact faces 10, 10′ are inclined in an orientation parallel to the vertebral body surfaces 30, 30′, such that the contact face 10, 10′ has its maximum extent.

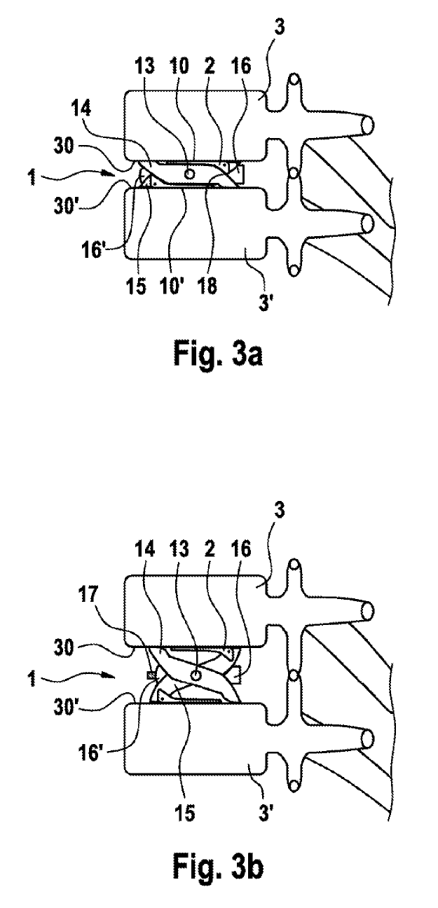

FIGS. 3a and 3b show the spreading-open of an intervertebral implant 1 with a bridge element 2 arranged at the upper vertebra 3. For this purpose, the intervertebral implant 1 may be arranged in the closed state between the vertebrae 3, 3′. When it has reached the intended position, the expansion device of the intervertebral implant 1 is actuated with the aid of the actuating element 12, such that the intervertebral implant 1 is expanded.

The intervertebral implant 1 can likewise be inserted in a position rotated 90° about the securing element 17. The actuation of the actuating element 12 then results in a spreading-open of the intervertebral implant 1 along the vertebral body surfaces 30, 30′.

The intervertebral implant 1 moreover has a strike face 11, which is designed to take up hammer blows. The strike face 11 is arranged on the widening component 16, 16′ on which the actuating element 12 is arranged. The intervertebral implant 1 can thus be inserted into an intervertebral space by means of a hammer. Moreover, by being arranged on the widening component 16, 16′ on which the strike face 11 is arranged, the actuating element 12 remains accessible from the outside and can be further actuated.

The widening component 16, 16′ that has no strike face 11 comprises a rounded rear wall 24. The rear wall 24 lies opposite the wedge tip 18, 18′ of the widening component 16, 16′. The insertion of the intervertebral implant 1 into the space between the vertebrae 3, 3′ is facilitated by the rounding of the rear wall 24.

The intervertebral implant 1 advantageously has a total length of between 15 mm and 30 mm, preferably 23 mm. Moreover, the intervertebral implant 1 advantageously has a height of between 7 mm and 12 mm, preferably 7 mm. It is also expedient that the intervertebral implant has a width of between 7 mm and 20 mm. During implantation, intervertebral implants with these dimensions require only small incisions to be made for placing them between the vertebrae 3, 3′.

The expansion device is moreover designed such that the contact faces 16, 16′ can be spaced apart from each other by up to 14 mm. Moreover, the intervertebral implant is made of titanium, CoCr or PEEK.

1. An intervertebral implant comprising at least two opposite contact faces, directed away from each other, for placement on mutually facing vertebral body surfaces of two adjacent vertebrae, wherein a distance between the two contact faces is modifiable by an expansion device which is arranged between the contact faces and has at least one actuating element, wherein the expansion device has at least two pivotable lever elements which are connected to each other like scissors via a pivot pin, and the expansion device comprises at least one widening component, which is actuatable by the actuating element and is configured to spread open the lever elements, and at least one securing element that is configured to fix a minimum spreading-open of the lever elements.

2. The intervertebral implant of claim 1, wherein the pivot pin divides the lever elements into two halves of equal length.

3. The intervertebral implant of claim 1, wherein the pivot pin divides the lever elements into two halves of different length.

4. The intervertebral implant of claim 1, wherein the securing element and the actuating element are formed in one piece.

5. The intervertebral implant of claim 1, wherein the securing element is designed a screw and the actuating element is a head of the screw, and the widening component is configured as at least two wedges connected to the screw as a spindle drive, wherein the at least two wedges are arranged on opposite sides of the pivot pin between the lever elements, and the wedges each have a wedge tip, the wedge tips pointing in opposite directions.

6. The intervertebral implant of claim 1, wherein at least one contact face is arranged on a bridge element which is arranged on one of the at least two lever elements by a fixed bearing, wherein the bridge element is arranged on another of the at least two lever elements by a floating bearing.

7. The intervertebral implant of claim 6, wherein the floating bearing is configured as a slideway.

8. The intervertebral implant of claim 6, wherein, when the implant is implanted in a closed state, the bridge element lies flush on the intervertebral implant.

9. The intervertebral implant of claim 6, wherein, when the implant is implanted in a closed state, the lever elements and the bridge elements form at least one lateral face of the intervertebral implant that is closed.

10. The intervertebral implant of claim 9, wherein the at least one lateral face of the intervertebral implant, in the spread-open state of the intervertebral implant, has apertures.

11. The intervertebral implant of claim 1, wherein the intervertebral implant has a total length of between 15 and 30 mm.

12. The intervertebral implant of claim 1, wherein the intervertebral implant has a height of between 7 and 12 mm.

13. The intervertebral implant of claim 1, wherein the intervertebral implant has a width of between 7 and 20 mm.

14. The intervertebral implant of claim 1, wherein the contact faces can be spaced apart from each other by up to 14 mm.

15. The intervertebral implant of claim 1, wherein at least one of the contact faces has a structured surface.

16. The intervertebral implant of claim 1, wherein a part of at least one of the contact faces is arranged at an end region of at least one of the lever elements and, when the intervertebral implant is in the closed state, the part of at least one of the contact faces is inclined about a central axis of the intervertebral implant.

17. The intervertebral implant of claim 16, wherein the angle of the inclination of the part of at least one of the contact faces is 5° to 30°.

18. The intervertebral implant of claim 1, further comprising a strike face, configured as an impact face for hammer blows.

19. The intervertebral implant of claim 1, wherein the intervertebral implant is made of titanium, a titanium alloy, CoCr or PEEK.

20. A set of intervertebral implants, wherein each intervertebral implant comprises at least two opposite contact faces, directed away from each other, for placement on mutually facing vertebral body surfaces of two adjacent vertebrae, wherein a distance between the two contact faces is modifiable by an expansion device which is arranged between the contact faces and has at least one actuating element, wherein the expansion device has at least two pivotable lever elements which are connected to each other like scissors via a pivot pin that divides each lever element into two lever arms, and the expansion device comprises at least one widening component, which is actuatable by the actuating element and is configured to spread open the lever elements, and at least one securing element that is configured to fix a minimum spreading-open of the lever elements,
wherein the intervertebral implants differ in a length ratio between the two lever arms of at least one lever element.

21. (canceled)

22. The intervertebral implant of claim 17, wherein the angle of the inclination is 10° to 20°.
